(19) United States
(12) Patent Application Publication
(10) Pub. No.: US 2021/0186336 A1
(43) Pub. Date: Jun. 24, 2021

(54) DEVICE FOR DETECTING PHYSICAL AND PHYSIOLOGICAL PARAMETERS OF A USER

(71) Applicant: TECNIMED S.R.L., Vedano Olona (VA) (IT)

(72) Inventors: Francesco BELLIFEMINE, Varese (IT); Cinzia BELLIFEMINE, Varese (IT); Andrea CERATI, Vedano Olona (VA) (IT); Iolanda SCIBELLI, Vedano Olona (VA) (IT)

(21) Appl. No.: 16/756,840

(22) PCT Filed: Oct. 12, 2018
(86) PCT No.: PCT/IB2018/057920

§ 371 Date: Apr. 16, 2020

(30) Foreign Application Priority Data
Oct. 16, 2017 (IT) 102017000116553

(51) Int. Cl. A61B 5/01; A61B 5/00; G01K 13/20

(52) U.S. Cl. CPC A61B 5/01; A61B 5/681; A61B 2560/0252; A61B 5/6807; G01K 13/20

(57) ABSTRACT

The present invention relates to a device (1) for detecting parameters of a user (3), provided with a wearable support (2) having a first surface (2a) configured for contacting a body surface of the user (3); the support (2) has a second surface (2b) opposed to the first surface (2a) and comprises a first temperature sensor (10) configured for emitting a signal representative of a temperature at the first surface (2a) of the support (2), and a second temperature sensor (11) configured for emitting a signal representative of a temperature at the second surface (2b) of the support (2). A control unit (19) is configured for receiving as an input the signals emitted by the first and second temperature sensors (10, 11) and for estimating a value representative of an internal body temperature of the user (3) as a function of both the signals emitted by the first and the second temperature sensors (10, 11) and/or estimating a value representative of a room temperature as a function of both the signals emitted by the first and second temperature sensors (10, 11).

### FIELD OF THE INVENTION

The present invention relates to a device wearable by a user for detecting physiological and/or physical parameters, wherein the device may be, for example, a bracelet. The device comprises a plurality of sensors for monitoring and transmitting information about one or more physiological parameters of the wearing user. The device finds application in all the sectors that contemplate a monitoring of the physiological parameters of the user, as well as physical parameters such as room temperature and of the wearing user. Furthermore, the device finds application in all the sectors that contemplate a remote monitoring and transmission of data concerning physical and physiological parameters to an external device, wherein the latter or the detection device can emit an alarm signal in case of danger on the base of the data collected. By way of example, the present invention may be used in the sales of articles for children and infants, in particular in order to try to avoid SIDS (Sudden Infant Death Syndrome) or to prevent the inattention to of the infant in the car.

In addition, the wearable device may also be used to monitor the user's position and/or distance (e.g., a child) from the remote control device (e.g., a parent's cell phone) and to control the surface temperature or the relative temperature of the wearing user.

### Background of the Invention

It is known to use detecting devices, such as bracelets or anklets, that can be worn by a user comprising a plurality of sensors adapted to monitor physiological and/or physical parameters. In particular, devices are known for detecting a variety of parameters by means of internal temperature sensors, room temperature sensors, motion sensors such as accelerometers, heart rate sensors, heartbeat presence and rate sensors, sensors for monitoring the concentration of oxygen in the blood, and GPS sensors. Wearable detecting devices are also known which comprise means for transmitting data remotely to an external device.

It is also known the possibility of equipping said detecting devices with loudspeakers, adapted to emit an acoustic signal if one or more physiological or physical parameters are evaluated outside a predetermined confidence interval.

A first example, described in patent application GB2487426A, relates to a wearable monitoring system, specific to avoid the infant's death in the crib (SIDS) and wearable by the child as a bracelet. The latter includes temperature and motion sensors, heart rate electrodes, baby body temperature and room temperature sensors. It is also configured for transmitting the data detected via Bluetooth. The invention is also optionally provided with a microphone for recording sounds.

However, it is noted that the detecting device described in patent application GB2487426A does not allow monitoring the concentration of oxygen in the blood or its geographical position by means of a GPS system. Moreover, said device does not provide the possibility of providing an accurate estimate of body temperature.

Also, it does not allow the presence or not of a breathing activity of the infant to be checked, or to set confidence intervals for each parameter detected in order to launch an alarm if one or more parameters are outside the predetermined interval. Furthermore, it does not allow a variable confidence interval to be defined according to the combination of the values of each parameter detected.

A further U.S. patent application US2015374293A1 describes an anklet also comprising a plurality of sensors for detecting parameters such as the infant's body temperature, movement, heartbeat, oxygenation. A remote signal transmission system allows the data to be sent to an external device. The invention also allows checking the presence of a heartbeat within a predetermined interval. However, even this patent application does not allow an accurate estimate of the infant's body temperature.

### OBJECT OF THE INVENTION

The object of the present invention therefore is to substantially solve the drawbacks and/or limitations of the above prior art.

A first object of the invention is to provide a wearable detecting device, in particular a bracelet or anklet, which can ensure a more accurate measurement, with respect to the aforementioned documents, of the room temperature and/or body temperature of the user. In particular, it is an object of the invention to determine the body temperature of the user by using at least two temperature sensors, compensating the body temperature value as a function of an external temperature. Furthermore, the object of the invention is to also have a reliable reading of the room temperature, despite the device and therefore the sensor being close to a heat source (heat of the wearing user).

A further object of the invention is to provide a detecting device which allows a plurality of physiological parameters of the user to be monitored, in particular the presence of a movement, the heartbeat rate, the breathing rate, and blood oxygenation.

Another object of the invention is to provide a device capable of providing an indication of its geographical position by means of a GPS and/or GNSS (Global Navigation Satellite System) sensor. Furthermore, it is an object of the invention to allow remote sending of the data acquired through the plurality of sensors to an external device.

An additional object of the invention is to provide a device capable of setting one or more confidence intervals for each physical or physiological parameter detected and of defining a dangerous condition if one or more of said parameters are outside the predetermined confidence interval. The danger condition therefore allows emit a sound or vibration alarm signal to be emitted by means of a loudspeaker or vibrodine placed on the detecting device and/or on the external device.

Furthermore, it is an object of the invention to provide a device capable of defining a variable confidence interval according to the combination of the values of each parameter detected.

### SUMMARY

Aspects of the invention according to the present invention are described below.

In a 1st aspect, a device (1) for detecting parameters of a user (3) is provided, comprising:
a support (2) wearable by the user (3) and exhibiting a first surface (2a) configured for facing, and optionally contacting, a body surface of the user (3), said support (2) exhibiting at least one second surface (2b) distanced from, and opposite to, the first surface (2a),
the wearable support (2) comprising:
   at least one first temperature sensor (10) configured for emitting at least one signal representative of a temperature at the first surface (2a) of the support (2), and
   at least one second temperature sensor (11) configured for emitting at least one signal representative of a temperature at the second surface (2b) of the support (2),
at least one control unit (19) configured for:
   receiving as an input at least the signals emitted by the first and second temperature sensors (10, 11),
   estimating a value representative of an internal body temperature of the user (3) as a function of both the signals emitted by said first and second temperature sensors (10, 11) and/or estimating a value representative of a room temperature as a function of both the signals emitted by said first and second temperature sensors (10, 11).

In a 2nd aspect according to the preceding aspect, the control unit (19) is configured for determining the temperature values at the first and/or second surface (2a, 2b) as a function of the signals emitted by the first and second temperature sensors (10, 11).

In a 3rd aspect according to the preceding aspect, the temperature value of the first surface (2a) approaches a surface body temperature of the user.

In a 4th aspect according to the aspect 2 or 3, the temperature value of the second surface (2b) approaches a room temperature.

In a 5th aspect according to any one of the preceding aspects, the value representative of an internal body temperature of the user (3) is estimated by multiplying a temperature value as a function of the signal emitted by the first temperature sensor (10) by a coefficient dependent on a temperature value as a function of the signal emitted by the second temperature sensor (11).

In a 6th aspect according to any one of the preceding aspects 2 to 5, the value representative of an internal body temperature of the user is estimated by multiplying the temperature value at the first surface (2a) by a coefficient dependent on the temperature value at the second surface (2b) of the support (2).

In a 7th aspect according to any one of the preceding aspects, the value representative of a room temperature determined by the second temperature sensor (11) is a function of a surface body temperature value of the user determined by said first temperature sensor (10).

In an 8th aspect according to any one of the preceding aspects, the value representative of a room temperature determined by the second temperature sensor (11) is corrected by a corrective factor.

In a 9th aspect according to any one of the preceding aspects, the value representative of a room temperature determined by the second temperature sensor (11) is corrected by multiplying the temperature determined as a function only of the signal of the second temperature sensor (11) by a corrective factor.

In a 10th aspect, optionally according to any one of the preceding aspects, a device (1) for detecting
parameters of a user (3), is provided, comprising:

a support (2) wearable by the user (3) and exhibiting a first surface (2a) configured for facing, and optionally contacting, a body surface of the user (3), said support (2) exhibiting at least one second surface (2b) distanced from, and opposite to, the first surface (2a),
the wearable support (2) comprising at least one first temperature sensor (10) configured for emitting at least one signal representative of a temperature at the first surface (2a) of the support (2),
at least one control unit (19) configured for:
   receiving as an input at least the signal emitted by the first temperature sensor (10),
   determining a first magnitude as a function of the variation of the signal emitted by the first temperature sensor (10) in a predetermined time interval,
   comparing said first magnitude with at least one first threshold,
   outputting, as a function of said comparison, a respective alarm signal.

In an 11th aspect according to the preceding aspect, the support (2) comprises at least one second temperature sensor (11) configured for emitting at least one signal representative of a temperature at the second surface (2b) of the support (2).

In a 12th aspect according to the preceding aspect, the control unit (19) is configured for receiving as an input at least the signal emitted by the second temperature sensor (11).

In a 13th aspect according to the aspect 11 or 13, the control unit (19) is configured for determining a second magnitude as a function of the variation of the signal emitted by the second temperature sensor (11) in a predetermined time interval.

In a 14th aspect according to the aspect 11 or 12 or 13, the control unit (19) is configured for defining the first threshold according to said second magnitude.

In a 15th according to the aspect 10, the support (2) comprises at least one second temperature sensor (11) configured for emitting at least one signal representative of a temperature at the second surface (2b) of the support (2),

wherein the control unit (19) is configured for:
receiving as an input at least the signal emitted by the second temperature sensor (11),
determining a second magnitude as a function of the variation of the signal emitted by the second temperature sensor (11) in a predetermined time interval,
comparing said second magnitude with at least one second threshold,
outputting, as a function of said comparison of the first magnitude with the first threshold and of the second magnitude with the second threshold, a respective alarm signal.

In a 16th aspect according to any one of the preceding aspects, the control unit (19) is configured for receiving, at a time instant T, from an auxiliary device, for example a thermometer, an internal body temperature value detected at the instant T′ on the user (3).

In a 17th aspect according to the preceding aspect, in which the control unit (19) is configured for determining a value of the internal body temperature of the user (3) based on the signal from the first temperature sensor (10), said temperature value being corrected as a function of the internal body temperature value received by the auxiliary device.

In an 18th aspect according to any one of the preceding aspects, the control unit (19) is configured for receiving, at a time instant T, from an auxiliary device, for example a thermometer, a detected room temperature value at the instant T′ in the environment wherein the user (3) is located.

In a 19th aspect according to the preceding aspect, the control unit (19) is configured for determining a value of the room temperature based on the signal coming from the second temperature sensor (11).

In a 20th aspect according to the preceding aspect, said temperature value is corrected as a function of the room temperature value received by the auxiliary device.

In a 21st aspect, optionally according to any one of the preceding aspects, a device (1) for detecting parameters of a user (3), is provided, comprising:
a support (2) wearable by the user (3) and exhibiting a first surface (2a) configured for facing, and optionally contacting, a body surface of the user (3),
said support (2) exhibiting at least one second surface (2b) distanced from, and opposite to, the first surface (2a),
the wearable support (2) comprising at least one of:
   a sensor configured for emitting a signal representative of the breathing rate;
   a sensor configured for emitting a signal representative of the presence or absence of a breathing process;
   an accelerometer sensor (13) configured for emitting at least one signal representative of a movement;
at least one control unit (19) configured for:
   receiving as an input at least the signals emitted by at least one sensor,
   determining, as a function of the input signals, a signal representative of the breathing rate and/or a signal representative of the presence or absence of a breathing process.

In a 22nd aspect according to the preceding aspect, the at least one accelerometer sensor (13) is configured for emitting at least one signal representative of at least one of the rate and the presence or absence of a breathing process of the user (3).

In a 23rd aspect according to the aspect 21 or 22, the at least one accelerometer sensor (13) is configured for emitting at least one signal representative of at least one of the rate and the presence or absence of a heartbeat of the user (3).

In a 24th aspect according to any one of the preceding aspects, the wearable support (2) comprises at least one vibrodine (16) configured for generating a predetermined vibration.

In a 25th aspect according to the preceding aspect, the control unit (19) selectively actuates the vibrodine (16) after determining the signal representative of the breathing rate and/or the signal representative of the presence or absence of a breathing process.

In a 26th aspect according to the aspect 24 or 25, the control unit (19) actuates the vibrodine (16) in the absence of a breathing process or in the presence of a breathing rate less than a preset safety value.

In a 27th aspect according to any one of the preceding aspects, the support (2) is enveloping.

In a 28th aspect according to any one of the preceding aspects, the support (2) is enveloping and comprises a first and a second coupling portion (30, 31) positioned at opposite ends of the support (2) itself and configured for cooperating with each other to define at least one closed condition.

In a 29th aspect according to any one of the preceding aspects, the support (2) is configured for firmly abutting at least a body portion of the user (3).

In a 30th aspect according to any one of the preceding aspects 28 or 29, the first coupling portion (30) removably couples to the second coupling portion (31) for example by Velcro®, button, buckle or optionally at least partially elastic strap coupling.

In a 31st aspect according to any one of the preceding aspects, the support (2) is enveloping and defines a closed profile and is elastically deformable to allow wearability by the user (3).

In a 32nd aspect according to any one of the preceding aspects 1 to 30, wherein the support (2) is enveloping and defines an open annular profile with the free ends facing.

In a 33rd aspect according to any one of the preceding aspects, the support is substantially rigid and has a degree of elastic deformability sufficient to allow wearability by the user.

In a 34th aspect according to any one of the preceding aspects, the support (2) is defined by at least one in the group of:
a bracelet;
an anklet;
a ring;
an abdominal support;
a collar;
a band.

In a 35th aspect according to any one of the preceding aspects, the control unit (19) is configured for estimating the value representative of the body temperature of the user (3) as a function of the combination of a predetermined temporal duration of the signals emitted by the first and second temperature sensors (10, 11).

In a 36th aspect according to any one of the preceding aspects, the value representative of the body temperature is calculated as a function of the rate of variation of the signals emitted by the first and second temperature sensors (10, 11) in a predetermined time interval.

In a 37th aspect according to any one of the preceding aspects, the wearable support (2) comprises one or more biometric sensors (12).

In a 38th aspect according to the preceding aspect, said biometric sensors (12) being configured for emitting one or more signals representative of one or more physiological parameters of the user (3).
In a 39th aspect according to the preceding aspect, said physiological parameters of the user (3) being detectable at the first surface (2a) of the support (2).

In a 40th aspect according to any one of the preceding aspects 37 to 39, the control unit (19) is configured for:
receiving as an input said one or more signals representative of physiological parameters;
determining one or more values representative of the respective physiological parameters.

In a 41st aspect according to any one of the preceding aspects 37 to 40, the one or more biometric sensors (12) comprise at least one in the group of:
at least one sensor configured for emitting a signal representative of the heart rate;
at least one sensor configured for emitting a signal representative of the breathing rate;
at least one sensor configured for emitting a signal representative of the presence or absence of a breathing process;
at least one sensor configured for emitting a signal representative of the blood oxygen concentration;
at least one sensor configured for emitting a signal representative of the blood CO₂ concentration.

In a 12th aspect according to the preceding aspect, the control unit (19) is configured for receiving as an input at least one of:
a signal representative of the heart rate;
a signal representative of the breathing rate;
a signal representative of the presence of a breathing process;
a signal representative of the blood oxygen concentration;

In a 43th aspect according to the aspect 41 or 42, said control unit (19) being configured for calculating respectively at least one of:
a value representative of a time heart rate;
a value representative of the time breathing rate;
a value representative of the presence of a breathing process;
a value representative of the blood oxygen concentration;
a value representative of the blood CO₂ concentration.

In a 44th aspect according to any one of the preceding aspects, the control unit (19) is configured for comparing the value of the internal body temperature with a predetermined value, for example 37°, to determine a potential feverish situation of the user.

In a 45th aspect according to the aspect 43 or 44, the control unit (19) verifies whether the value representative of the heartbeat rate exceeds or not a predetermined threshold value for the beat rate.

In a 46th aspect according to the aspect 43 or 44 or 45, the control unit (19) verifies whether the value representative of the breathing rate exceeds or not a predetermined threshold value for the breathing rate.

In a 47th aspect according to any one of the aspects 44 to 46, the control unit (19) being configured for determining a feverish situation as a function of the outcome of both comparisons.

In a 48th aspect according to any one of the preceding aspects, the wearable support (2) comprises at least one accelerometer sensor (13) configured for emitting at least one signal representative of a movement.

In a 49th aspect according to the preceding aspect, the accelerometer sensor (13) is configured for emitting a signal representative of a movement in one or more directions and/or one or more rotations.

In a 50th aspect according to any one of the aspects 48 or 49, the accelerometer sensor (13) is configured for emitting at least one signal representative of at least one of the rate and the presence or absence of a breathing process of the user (3).

In a 51st aspect according to any one of the aspect 48 or 49 or 50, the at least one accelerometer sensor (13) is configured for emitting at least one signal representative of at least one of the rate and the presence or absence of a heartbeat of the user (3).

In a 52nd aspect according to any one of the aspects 48 to 51, the accelerometer sensor (13) is configured for emitting a signal representative of an acceleration, optionally a triaxial acceleration.

In a 53rd aspect according to any one of the aspects 48 to 52, the wearable support (2) further comprises an auxiliary accelerometer sensor.

In a 54th aspect according to the preceding aspect, the control unit (19) is configured for receiving the signals coming from the accelerometer sensors and process them jointly, obtaining at least one signal representative of at least one of the rate and the presence or absence of a heartbeat of the user (3).

In a 55th aspect according to any one of the aspects 48 to 54, wherein the accelerometer sensor (13) is positioned at the first surface (2a) of the wearable support (2) to contact the user (3) when the device is in use.

In a 56th aspect according to any one of the aspects 48 to 55, the auxiliary accelerometer sensor is positioned on the support (2), at a distance from the first surface (2a) and not rigidly coupled to the accelerometer sensor (13).

In a 57th aspect, a device is provided according to any one of the preceding aspects, further comprising at least one blood saturation/oxygenation sensor (21).

In a 58th aspect according to the preceding aspect, the blood saturation/oxygenation sensor (21) is configured for emitting at least one signal representative of the saturation/oxygenation of the user (3).

In a 59th aspect according to the aspect 57 or 58, said blood saturation/oxygenation sensor (21) being distinct from the support (2) and connectable by cable (22) or wirelessly to the control unit (19).

In a 60th aspect, a device is provided according to any one of the preceding aspects, comprising at least one blood CO₂ concentration sensor.

In a 61st aspect according to the preceding aspect, the blood CO₂ concentration sensor is configured for emitting at least one signal representative of the blood CO₂ concentration of the user (3).

In a 62nd aspect according to the aspect 60 or 61, the blood CO₂ concentration sensor is distinct from the support (2) and connectable by cable or wirelessly to the control unit (19).

In a 63rd aspect according to any one of the preceding aspects, wherein the control unit (19) is configured for storing the signal representative of at least one between the frequency and the presence or absence of a breathing process of the user (3) in a memory connected to the control unit itself.

In a 64th aspect according to the preceding aspect, the control unit (19) is configured for graphically representing the time trend of the signal representative of at least one between the frequency and presence or absence of a breathing process in the user (3) on a display connected to the control unit (19) itself.

In a 65th aspect according to any one of the aspects 48 to 64, the control unit (19) is configured for:
receiving as an input at least the at least one signal representative of a movement emitted by the accelerometer sensor (13),
determining a value representative of a movement of the support (2) of the detecting device (1).

In a 66th aspect according to the preceding aspect, the value representative of a movement is defined by at least one between acceleration, speed or displacement imposed on the support (2) of the detecting device (1).

In a 67th aspect according to any one of the aspects 48 to 66, wherein the control unit (19) is configured for comparing the value representative of a movement of the support (2) of the detecting device (1) with at least one threshold value.

In a 68th aspect according to the preceding aspect, the control unit (19) is configured for determining a feverish situation also as a function of the result of the comparison between the value representative of a movement and the threshold value thereof.

In a 69th aspect according to any one of the preceding aspects, the wearable support (2) comprises at least one position sensor (14).

In a 70th aspect according to the preceding aspect, the position sensor (14) defines a global positioning system, in particular the position sensor (14) being a GPS (Global Positioning System) or GNSS (Global Navigation Satellite System) sensor or a combination thereof.

In a 71th aspect according to any one of aspects 69 or 70, the position sensor (14) is configured for emitting a signal representative of a geographical position of the support (2).

In a 72nd aspect according to any one of the aspects 69 to 71, the control unit (19) is configured for:
receiving as an input at least one signal representing the position of the support (2) emitted by the position sensor (14);
determining a value representative of the position of the support (2) of the detecting device (1).

In a 73th aspect according to the preceding aspect, the control unit (19) is configured for determining the value representative of the geographical position in Cartesian or polar coordinates of the support (2) of the detecting device (1).

In a 74th aspect according to the preceding aspect, the value representative of the position has a tolerance of less than 30 m, optionally a tolerance of less than 10 m, in particular with a tolerance of less than 1 m.

In a 75th aspect according to any one of the preceding aspects, the wearable support (2) comprises signal transmission means (15).

In a 76th aspect according to the preceding aspect, the transmission means (15) are configured for remotely transmitting data.

In a 77th aspect according to aspect 75 or 76, the transmission means (15) are configured for remotely transmitting at least one in the group between:
the temperature values at the first and second surfaces (2a, 2b) of the support (2);
the value representative of the body temperature;
the value representative of a room temperature;
the value representative of the geographic position of the support (2);
the value representative of a movement;
the value representative of the heart rate;
the value representative of the breathing rate;
the value representative of the presence of a breathing process;
the value representative of the blood oxygen concentration.

In a 78th aspect according to any one of the aspects 75 to 77, the transmission means (15) comprise at least one in the group between:
a radio frequency transmission system;
an RFID (Radio-Frequency IDentification) system;
a Wi-Fi system, in particular based on the IEEE 802.11 standard specifications;
a Bluetooth system.

In a 79th aspect according to any one of the preceding aspects, the wearable support (2) comprises at least one screen (20) configured for allowing the display of at least one of:
the temperature values at the first and second surfaces (2a, 2b);
the value representative of the body temperature;
the value representative of a room temperature;
the value representative of the position of the support (2);
the value representative of a movement;
the value representative of the heart rate;
the value representative of the breathing rate;
the value representative of the presence of a breathing process;
the value representative of the blood oxygen concentration.

In an 80th aspect according to any one of the preceding aspects, the control unit (19) is configured for defining one or more confidence intervals.

In an 81st aspect according to the preceding aspect, the control unit (19) being configured for:
comparing at least one value representative of one or more parameters of interest with a respective confidence interval;
as a function of said comparison, defining:
   a safety condition, wherein the at least one representative value falls into the respective confidence interval;
   a danger condition, wherein the at least one representative value exceeds the confidence interval.

In an 82nd aspect according to any one of aspects 80 or 81, the control unit (19) being configured for emitting, in the dangerous condition, a respective alarm signal.

In an 83rd aspect according to any one of aspects 80 to 82, the control unit (19) defines one or more confidence intervals, optionally predetermined, relative to each value representative of the respective acquired parameter.

In an 84th aspect according to any one of the aspects 80 to 82, the control unit (19) defines one or more confidence intervals as a function of the combination of, optionally of the interaction between, the values representative of the acquired parameters.

In an 85th aspect according to any one of aspects 81 to 84, the wearable support (2) comprises at least one vibrodine (16) configured for generating, in the dangerous condition, a predetermined vibration.

In an 86th aspect according to any one of aspects 81 to 85, the wearable support (2) comprises at least one loudspeaker (17) configured for emitting, in the dangerous condition, a respective sound signal.

In an 87th aspect according to any one of the preceding aspects, the wearable support (2) comprises at least one vibrodine (16) configured for generating a predetermined vibration.

In an 88th aspect according to any one of the preceding aspects, the wearable support (2) comprises at least one loudspeaker (17) configured for emitting a respective sound signal.

In an 89th aspect according to any one of the preceding aspects, the wearable support (2) comprises at least one battery (18), optionally rechargeable.

In a 90th aspect according to the preceding aspect, the battery (18) supplies the control unit (19) and the one or more sensors of the support (2).

In a 91st aspect according to aspect 89 or 90, the battery (18) supplies at least one of:
the first and the second temperature sensors (10, 11);
at least one biometric sensor (12);
at least one accelerometer sensor (13);
at least one GPS position sensor (14);
the signal transmission means (15);
at least one vibrodine (16);
the at least one loudspeaker 17
the control unit (19);
the at least one screen (20).

In a 92nd aspect according to any one of the preceding aspects, the control unit (19) comprises at least a first controller (19a) engaged to the wearable support (2) and in connection with the first and the second temperature sensors (10, 11).

In a 93rd aspect according to the preceding aspect, the first controller (19a) being configured for:
receiving as an input at least the signals emitted by the first and second temperature sensors (10, 11),
estimating a value representative of a body temperature of the user as a function of both the signals emitted by said first and second temperature sensors (10, 11).

In a 94th aspect according to the aspect 92, the first controller (19a) being configured for:
receiving as an input at least the signals emitted by the first and second temperature sensors (10, 11),
estimating a value representative of a room temperature according to both the signals emitted by said first and second temperature sensors (10, 11).

In a 95th aspect according to aspect 92 or 93 or 94, wherein the first controller (19a) is connected to at least one of the biometric sensors (12).

In a 96th aspect according to the preceding aspect, the first controller (19a) being configured for:
receiving from one or more of the biometric sensors (12) one or more signals representative of at least one physiological parameter of the user (3);
determining one or more values representative of the respective physiological parameters.

In a 97th aspect according to any one of the aspects 92 to 96, the first controller (19a) is connected to the at least one accelerometer sensor (13) of the support (2).

In a 98th aspect according to the preceding aspect, the first controller (19a) being configured for:
receiving at least the at least one signal representative of a movement emitted by said accelerometer sensor (13),
determining a value representative of a movement of the support (2), optionally wherein said value representative of a movement is defined by at least one of an acceleration, speed or displacement of the support (2) of the detecting device (1).

In a 99th aspect according to aspect 97, the first controller (19a) being configured for:
receiving at least the at least one signal representative of a movement emitted by said accelerometer sensor (13),
determining a value representative of a movement of the support (2), optionally wherein said value representative of a movement is defined by at least one of an acceleration, speed or displacement of the support (2) of the detecting device (1).

In a 100th aspect according to any one of the aspects 92 to 99, the first controller (19a) is connected to the at least one position sensor (14) of the support (2).

In a 101st aspect according to the preceding aspect, the first controller (19a) being configured for:
receiving as an input at least one signal representing the position of the support (2) emitted by the position sensor (14);
determining a value representative of the position of the support (2) of the detecting device (1).

In a 102nd aspect according to aspect 100, the first controller (19a) being configured for:
receiving as an input at least one signal representing the position of the support (2) emitted by the position sensor (14);
determining a value representative of the position of the support (2) of the detecting device (1), in particular a value representative of the position in Cartesian or polar coordinates of the support (2), in particular wherein said value representative of the position has a tolerance of less than 30 m, optionally a tolerance of less than 10 m, in particular with a tolerance of less than 1 m.

In a 103rd aspect according to any one of the preceding aspects, the detecting device comprises at least one external device (100) spaced from the support (2).

In a 104th aspect according to the preceding aspect, the external device (100) comprises signal receiving means (101) configured for receiving remotely from the transmission means (15) of the wearable support (2) at least the value representative of the body temperature of the user (3).

In a 105th aspect according to the preceding aspect, the external device (100) comprises signal receiving means (101) configured for receiving remotely from the transmission means (15) of the wearable support (2) at least one in the group among:
the temperature values at the first and second surfaces (2a, 2b) of the support (2);
the value representative of a room temperature;
the value representative of the position of the support (2);
the value representative of a movement;
the value representative of the heart rate;
the value representative of the breathing rate;
the value representative of the presence of a breathing process;
the value representative of the blood oxygen concentration;
a respective alarm signal emitted by the control unit (19) of the detecting device during the danger condition.

In a 106th aspect according to any one of aspects 103 to 105, the control unit (19) comprises at least one second controller (19b) engaged to the external device (100) and in connection at least with the signal receiving means (101) of said external device (100).

In a 107th aspect according to the preceding aspect, the second controller (19b) being configured for receiving as an input at least one in the group between:
the value representative of the body temperature of the user (3);
the temperature values at the first and second surfaces (2a, 2b) of the support (2);
the signals emitted from the first and second temperature sensors (10, 11);

In a 108th aspect according to the preceding aspect, the second controller (19b) being configured for determining at least one in the group between:
a value representative of a body temperature of the user (3) as a function of both the signals emitted by said first and second temperature sensors (10, 11);
the temperature values at the first and second surfaces (2a, 2b).

In a 109th aspect according to aspect 107, the second controller (19b) being configured for:
estimating a value representative of a room temperature according to both the signals emitted by said first and second temperature sensors (10, 11); and/or
the temperature values at the first and second surfaces (2a, 2b).

In a 110th aspect according to any one of aspects 106 to 109, the second controller (19b) is configured for:
receiving at least at least one signal representative of one or more physiological parameters of the user (3) emitted by the at least one biometric sensor (12);
determining at least one value representative of the respective physiological parameters.

In a 111th aspect according to any one of aspects 106 to 110, the second controller (19b) is configured for receiving as an input, from the signal receiving means of the external device, at least one value representative of the physiological parameters detected by one or more of the biometric sensors (12) of the support (2).

In a 112th aspect according to any one of aspects 106 to 111 the second controller (19b) is configured for:
receiving as an input a signal representative of an acceleration emitted by the accelerometer sensor (13) of the support (2);
determining a value representative of an acceleration of the support (2).

In a 113th aspect according to any one of aspects 106 to 112, the second controller (19b) is configured for receiving as an input, from the signal receiving means (101) of the external device (100) a value representative of an acceleration of the support (2).

In a 114th aspect according to any one of aspects 106 to 113 the second controller (19b) is configured for:
receiving as an input a signal representative of a position of the support (2) emitted by the position sensor (14);
determining a value representative of a position of the support (2).

In a 115th aspect according to any one of aspects 106 to 114, the second controller (19b) is configured for receiving as an input, from the signal receiving means (101) of the external device (100) a value representative of a position of the support (2).

In a 116th aspect according to any one of aspects 103 to 115, the external device (100) comprises a position sensor (102), optionally a position sensor defining a global positioning system, in particular the position sensor (102) being a GPS (Global Positioning System) sensor or a GNSS (Global Navigation Satellite System) sensor.

In a 117th aspect according to the preceding aspect, the position sensor (102) being configured for emitting a signal representative of the geographical position of the external device (100).

In a 118th aspect according to any one of aspects 106 to 116, the second controller (19b) is configured for:
receiving as an input at least one signal representing the position of the external device (100) emitted by the position sensor (102);
determining a value representative of the geographical position of the external device (100), in particular a value representative of the geographical position in Cartesian or polar coordinates of the external device (100),
in particular wherein said value representative of the geographical position has a tolerance of less than 30 m, optionally a tolerance of less than 10 m, in particular with a tolerance of less than 1 m.

In a 119th aspect according to any one of aspects 106 to 118, the second controller (19b) is configured for defining one or more confidence intervals.

In a 120th aspect according to any one of aspects 106 to 119, said second controller (19b) being further configured for:
comparing at least one value representative of one or more parameters of interest, optionally received from the signal receiving means, with a respective confidence interval;
as a function of said comparison, defining:
   a safety condition, wherein the at least one representative value falls into the respective confidence interval;
   a danger condition, wherein the at least one representative value exceeds the confidence interval.

In a 121st aspect according to any one of aspects 106 to 120, the second controller (19b) being configured for emitting an alarm signal in the dangerous condition.

In a 122nd aspect according to any one of aspects 103 to 121, the external device (100) comprises, optionally bears, at least one in the group between:
at least one further screen (105) configured for displaying one or more values representative of the parameters of interest;
at least one further loudspeaker (104) configured for emitting a sound signal in the danger condition;
at least one further vibrodine (103) configured for generating, in the dangerous condition, a predetermined vibration.

In a 123rd aspect according to any one of aspects 106 to 122, the external device (100) comprises, optionally bears, at least one in the group between:
at least one further screen (105) configured for displaying one or more values representative of the parameters of interest;
at least one further loudspeaker (104) configured for emitting a sound signal;
at least one further vibrodine (103) configured for generating a predetermined vibration.

In a 124th aspect according to any one of the preceding aspects, the confidence interval relative to the value representative of the geographical position of the support (2) defines a safety area.

In a 125th aspect according to any one of aspects 92 to 124, the first controller (19a) of the support (2) is configured for:
comparing the value representative of the geographical position of the support (2) with the respective confidence interval defining the safety area;
defining the danger condition if the confidence interval defining the safety area does not include the value representative of the geographical position of the support (2).

In a 126th aspect according to any one of aspects 116 to 125, the confidence interval relative to the value representative of the geographical position of the external device (102) defines a relevance area, optionally said relevance area being defined as a function the geographical position of the external device (100) itself.

In a 127th aspect according to any one of aspects 106 to 126, the second controller (19b) of the external device (100) is configured for:
comparing the value representative of the geographical position of the support (2) with the confidence interval defining the relevance area;
defining the danger condition if the confidence interval defining the relevance zone does not include the value representative of the geographical position of the support (2).

In a 128th aspect according to aspect 126 or 127, the relevance zone defines an area limited to a maximum distance from the external device (100).

In a 129th aspect according to any one of the preceding aspects, the detection device (1) comprises storage means configured for recording the values representative of the parameters of interest.

In a 130th aspect according to any one of the preceding aspects, the external device (100) comprises storage means configured for recording the values representative of the parameters of interest.

### DESCRIPTION OF THE DRAWINGS

Some embodiments and some aspects of the invention will be described below with reference to the accompanying drawings, provided for indicative purposes only and therefore not limiting, wherein:

FIG. 1a shows a preferred schematic embodiment of a support of the detecting device according to the present invention;

FIG. 1b shows a schematic embodiment alternative to that in FIG. 1a of the support of the detecting device according to the present invention;

FIG. 2a shows a schematic embodiment of a support of the detecting device worn by a user;

FIG. 2b shows a variant embodiment of the support of the detecting device in FIG. 2a in which a further external sensor is present;

FIG. 3 shows a logic diagram of the detecting device comprising the support and the external device;

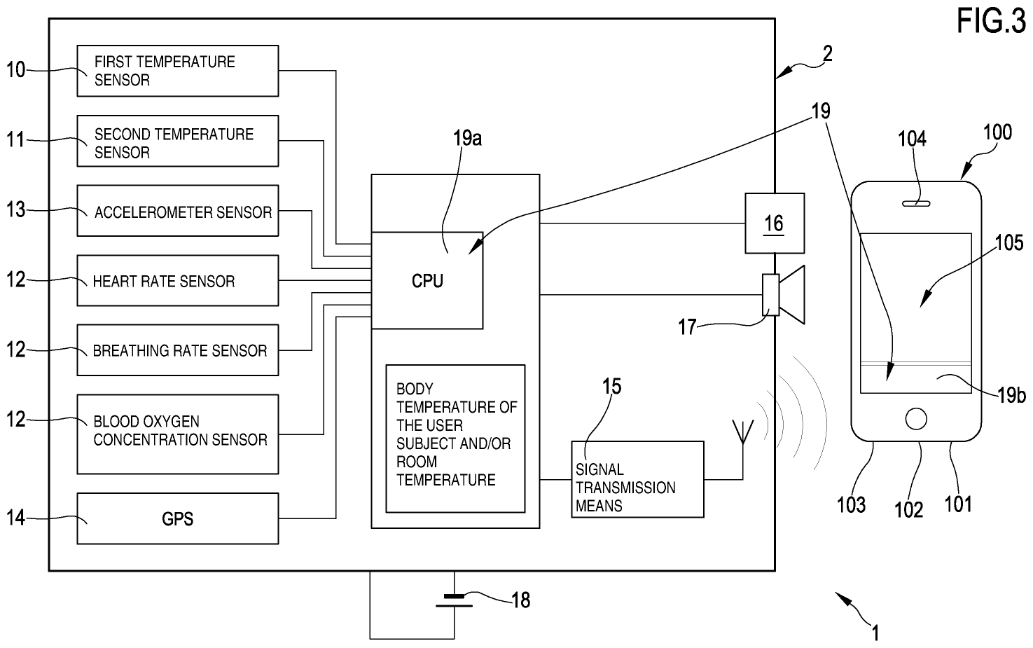

FIG. 4 shows a schematic representation of the time course of the breathing signal detected by an accelerometer sensor.

### DESCRIPTION

It should be noted that in the present detailed description, corresponding parts illustrated in the various figures are indicated with the same numerical references. The figures may illustrate the object of the invention by representations that are not in scale; therefore, parts and components illustrated in the figures relating to the object of the invention may relate solely to schematic representations.

The present invention relates to a device 1 for detecting parameters of a user 3. In particular, the detecting device 1 is adapted to monitor, analyze and manage physiological parameters of a user 3 such as temperature, heartbeat, blood oxygenation, breathing rate, etc.; the device is also adapted to monitor, analyze and manage external physical parameters, such as temperature, humidity, position, etc.

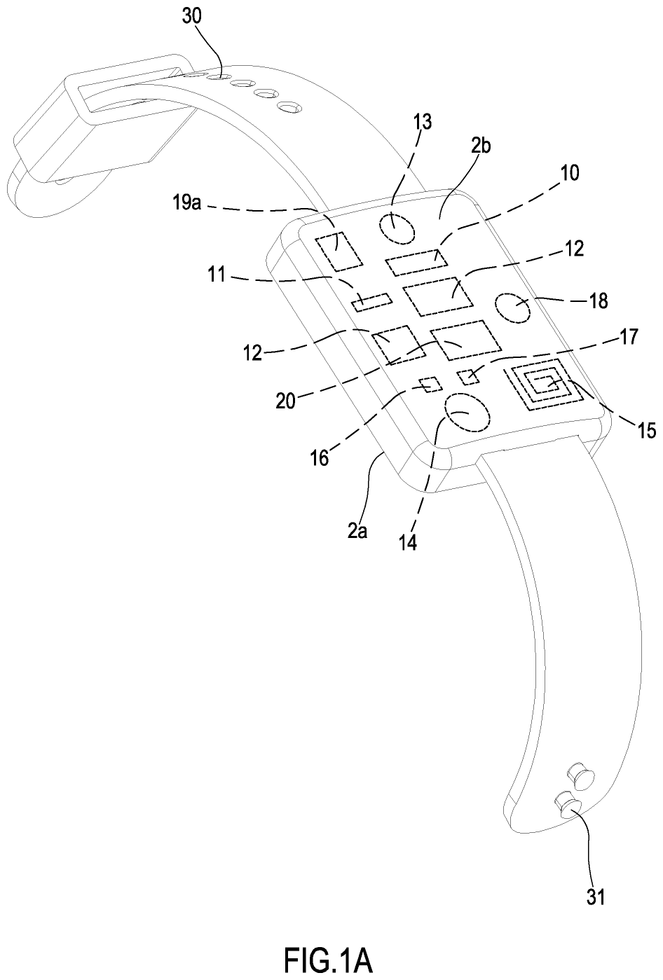

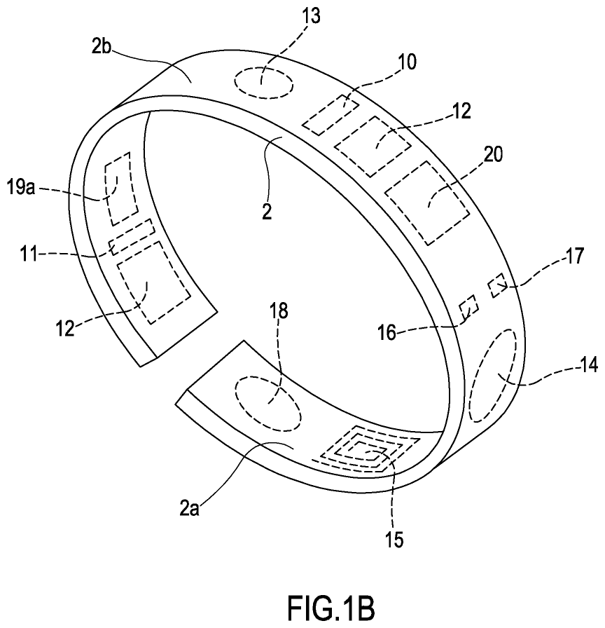

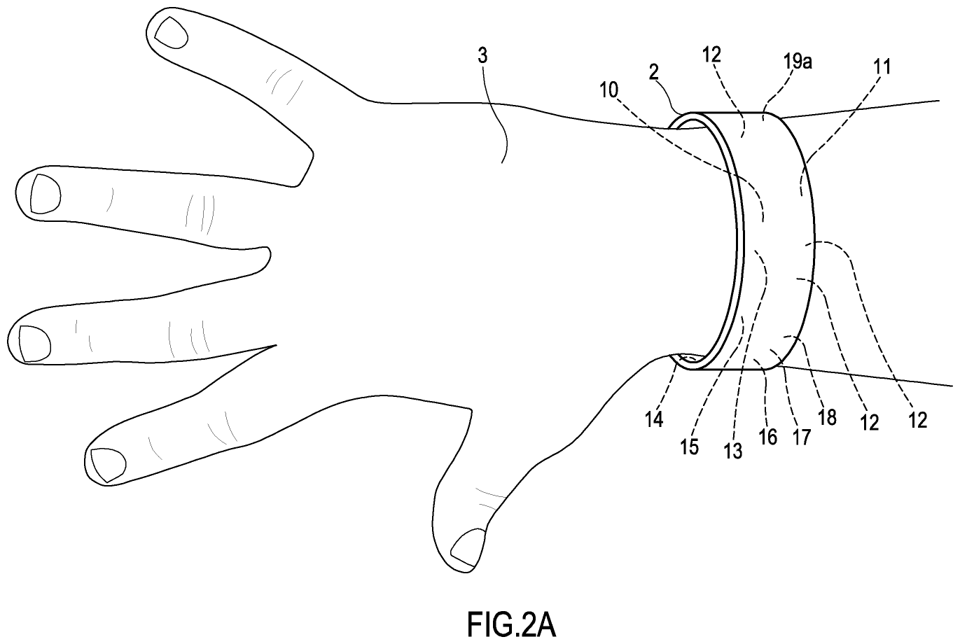

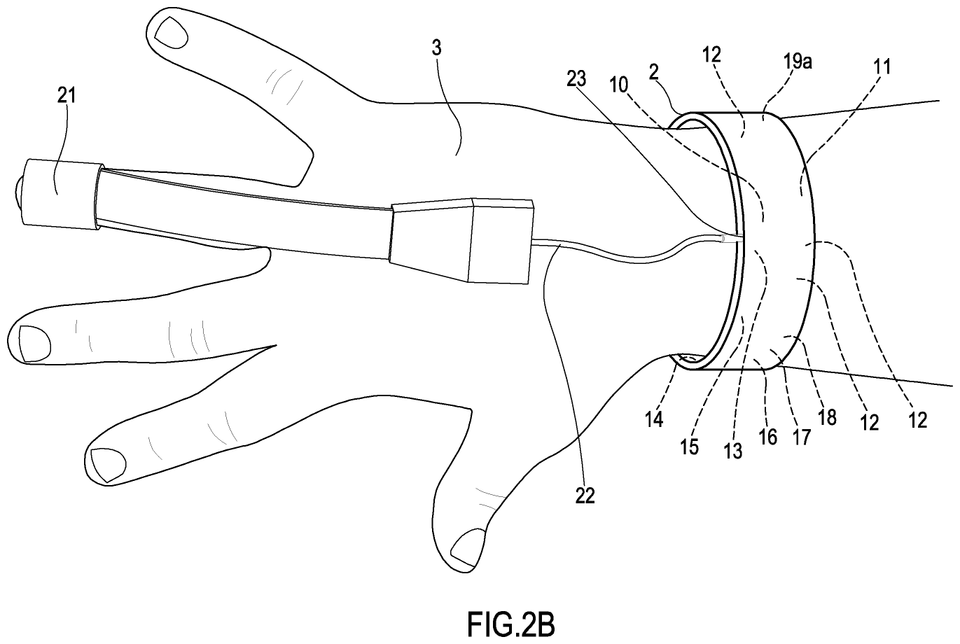

The detecting device 1 comprises a support 2, shown schematically in FIGS. 1a, 1b and 2, which can be worn by the user 3. The support 2 is enveloping, in particular around at least one body portion of the user 3 to come into contact with the latter, and comprises, in an embodiment shown in FIG. 1a, a first and a second coupling portion 30, 31 positioned at opposite ends of the support 2 itself (not shown in the accompanying figures). The coupling portions 30, 31 are configured for cooperating with each other to define at least one closed condition, during which the support 2 is configured for steadily abutting at at least one body portion of the user. The first coupling portion 30 is configured for removably coupling to the second coupling portion 31 of the same support 2: in a preferred embodiment, the first and the second coupling portion 30, 31 define a closing system selected from Velcro® type, button, buckle, strap or a combination thereof. The first and second coupling portions 30, 31 define the coupling system of the support 2. In the latter embodiment, the support 2 defines an open profile in an open condition of the coupling system while it defines a closed profile in a closed condition of the same coupling system.

In a further embodiment, the support 2 is enveloping and defines a closed profile: moreover, the support 2 is elastically deformable to allow the user 3 to wear it (for example a bracelet or an anklet, at least partially made of elastic material).

In an optional embodiment shown in FIG. 1b, the support 2 is enveloping and defines an open annular profile with the facing free ends: the support may be substantially rigid and/or have a degree of elastic deformability sufficient to allow wearability by the user.

The support 2 may be worn by a user (see FIG. 2) of any age and gender. In particular, the support 2 may be worn on the wrist, arm, alternately ankle, chest, hand, foot or neck. In detail, the support 2 may be a bracelet, an anklet 6, an abdominal support, a ring, a collar or a band.

As shown in FIGS. 1a and 1b, the support 2 has a first surface 2a (within the ring) configured for facing a body surface of the user 3: in particular, the first surface 2a is configured for contacting a body surface of the user 3. The support 2 also has at least a second surface 2b spaced from the first surface 2a: in particular, the second surface 2b is opposed to the first surface 2a of the support 2. Even in more detail, the second surface 2b is not configured for facing and/or contacting the body surface of the user 3 being facing away from the user when the device is worn.

It should be noted that an optimal contact between the support 2, in particular between the first surface 2a and the body portion (e.g. the arm) of the user is desirable in that it allows the biometric sensors present in the support and described hereafter to be able to better acquire the reference signals (such as heartbeat, surface body temperature or breathing). In this regard, the embodiment shown in FIG. 1a, as well as the one with a buckle and a perforated strap, appears to be optimal since the closure system can be adapted more precisely to the size of the arm. Also an elastic structure of a part of the support (such as the strap part) allows maintaining a constant contact and better detecting the signals. However, this does not exclude the possibility of adopting other embodiments, such as that illustrated in FIG. 1b. Also a support with a shape memory strap could be adopted without departing from the concept of the present invention; in this way, the strap would perfectly fit in use to the body portion of the user.

The support 2, according to each of the previously described embodiments, may be made of polymeric material, plastic, rubber or of a composite material such as, for example, glass or carbon fiber. Furthermore, the support 2 may be made of a metal material, in particular steel, titanium, copper, aluminum or an alloy thereof. Furthermore, the support 2 may be coated superficially with materials such as nickel, gold or platinum.

In a preferred embodiment, the detecting device 1 comprises a control unit 19, configured for receiving as an input and suitably managing one or more signals emitted by a plurality of sensors described in detail hereafter. In particular, the control unit 19 is of the digital or analog type and provides the possibility of having multiple inputs for receiving the signals, as well as a plurality of outputs so as to be able to transmit a modified signal following a predetermined analysis. The control unit 19 is configured for operating in a variable range of frequencies: in particular, the control unit 19 allows carrying out detection, sampling, data analysis or emitting signals over a wide range of operating frequencies. In detail, the sampling of the signals can occur at frequencies of between 10,000 Hz and 10\hat{ }-100 Hz, more particularly between 1000 Hz and 0.01 Hz, even more particularly between 100 Hz and 0.1 Hz.

In a preferred embodiment, the control unit 19 comprises a first controller 19a directly connected to the support 2 of the detecting device 1 and configured for receiving as an input one or more signals emitted by a plurality of sensors described in detail hereafter. In particular, the first controller 19a is of the digital or analog type and provides the possibility of having multiple inputs for receiving the signals, as well as a plurality of outputs so as to be able to transmit a modified signal following a predetermined analysis. The first controller 19a and/or the control unit 19 comprise a data memory device, in particular a digital memory of the type, for example and not limited thereto, hard disk, SD card, integrated memory, solid state memory.

The wearable support 2 comprises one or more biometric sensors 12 configured for emitting one or more signals representative of physiological parameters of the user, optionally detectable at the first surface 2a of the support 2. In detail, the biometric sensors are facing and/or in contact with at least one body surface of the user 3. In a preferred embodiment, the plurality of biometric sensors may comprise one or more sensors configured for emitting a signal representative of a heart rate, one or more sensors configured for emitting a signal representative of the breathing rate, one or more sensors configured for emitting a signal representative of the presence or absence of a breathing process, one or more sensors configured for emitting a signal representative of the blood oxygen concentration. The biometric sensors may be placed at the first or second surfaces 2a, 2b of the support 2, preferably at the first surface 2a facing the body surface of the user. Certain sensors, where appropriate, may be embedded within the support itself.

The control unit 19, in particular the first controller 19a, is configured for receiving as an input one or more signals representative of physiological parameters emitted by the biometric sensors and for determining, optionally calculating, one or more values representative of the respective physiological parameters. In detail, the control unit 19, in particular the first controller 19a, is configured for receiving as an input (at least one of):
a signal representative of the heart rate;
a signal representative of the breathing rate;
a signal representative of the presence of a breathing process;
a signal representative of the blood oxygen concentration.

Subsequently, the control unit 19, in particular the first controller 19a, is configured for calculating respectively:
a value representative of a time heart rate;
a value representative of the time breathing rate;
a value representative of the presence of a breathing process;
a value representative of the blood oxygen concentration.

In a specific embodiment (see FIG. 2b), the biometric blood oxygenation sensor 21 is positioned externally to the main body of the support 2 and connected to the body by a cable 22. Since the control of blood oxygenation is rather critical if done on the wrist or ankle, alternatively to the presence of a sensor of this type embedded in the main body of the support, it will be possible to connect the support 2 to the user's wrist or ankle, and connect a suitable oxygenation/saturation sensor 21, such as an oximeter, separated from the bracelet and which may be applied to the user's finger or foot; the connection may be (as shown) obtained by cable 22 by means of a socket 23 (for example jack type) on the main body of the support 2 to let out a cable which feeds the sensor itself and with which to receive reliable data. Another possibility is to adopt also (or as an alternative to the oximeter), a blood CO₂ concentration sensor. The same is useful for diagnosing problems that precede SIDS more easily. The simplest sensor may be of an optical type, such as a sensor consisting of a particular wavelength LED and a receiver of such light.

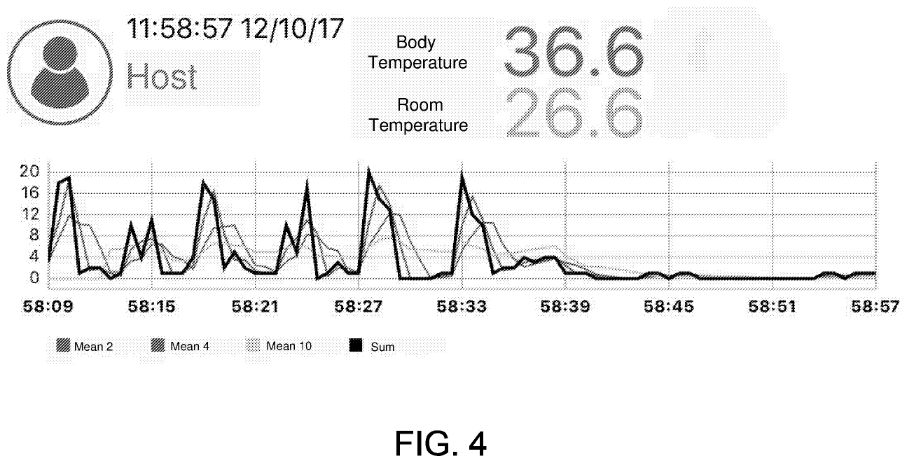

The wearable support 2 further comprises at least one accelerometer sensor 13 configured for emitting at least one signal representing a movement, in one or more directions and/or rotations, of the support 2 itself. In particular, in the condition in which the support 2 is worn by the user, the one or more accelerometer sensors are adapted to monitor the movement of the user him/herself. In particular, the accelerometer sensor 13 is configured for emitting a signal representative of an acceleration, mono axial, biaxial or triaxial. The acceleration signal emitted by the movement sensor may be used to also calculate the speed and position of the support 2 of the detecting device. It should be noted that the accelerometer sensor(s) may also be used to determine the breathing rate or the presence or absence of the breathing process. FIG. 4 illustrates a graphical representation of the breathing trend of a user detected by using the aforementioned accelerometer sensor 13. The various broken lines are the averages or the sum of the movements detected on the 3 axes of the sensor. The very evident peaks in the chart are the breathing acts. As illustrated, from minute 58 and 33 seconds, the breathing stops and a few seconds later (in this case about 18 seconds, but obviously this time may be changed) at exactly 11.58:51, the system goes into alarm by entering the last minute data in a non-volatile memory and in fact by taking a screen shot of the graph at the relevant moment.

Moreover, the accelerometer sensor(s) will be able to determine the presence of a heartbeat or blood flow in the veins in the vicinity of the sensor. Preferably, the support 2 comprises two (or even three, as explained hereafter) distinct accelerometer sensors 13, the first accelerometer sensor configured for determining the breathing rate or also the presence or absence of the breathing process, the second accelerometer sensor configured for determining the presence of heartbeat or blood flow in the veins in the vicinity of the sensor. The accelerometer sensor may be of the resistive or capacitive type or based on a piezoelectric or other effect, providing a variation of an electrical or capacitive resistance or of an electric potential difference. Said accelerometer sensor 13 may also be configured for emitting a reference gravity acceleration signal. The control unit 19, in particular the first controller 19a, is configured for receiving at least at least one signal representative of a movement emitted by the accelerometer sensor 13, and, subsequently, for determining a value representative of a movement of the support 2 of the detecting device. 1. In greater detail, the value representative of a movement defines at least one between acceleration, speed or displacement of the support 2 of the detecting device 1. In addition, the value representative of a movement defines a maximum distance traveled, a value representative of energy dissipated during the movement, or the jerk value defined as the variation rate of the acceleration value over time.

As mentioned, the accelerometer sensor may also (or only) be used for determining a parameter relative to the user's breathing in order to allow the control unit 19 to detect the vibrations transmitted to the body by breathing; in other words, it will be possible to determine the breathing rate by filtering it and recognizing the vibrations thereof with respect to those of the heartbeat, of the user's movement, of any further external disturbances. In this regard, the possible presence of two accelerometers could be very useful. In fact, the first accelerometer sensor 13 could by itself have problems to detect the breathing rate, or to understand if there is no breath, for example in the case where the user is in a moving car or on a wheelchair due to the vibrations generated by the movement. In this situation, the presence of a first accelerometer 13 on the surface 2a of the wearable support 2 in contact with the user's body would receive first signals; the presence of a second accelerometer 13 positioned for example at the surface 2b facing outwards, and mounted on disconnected circuits or in any case not rigidly connected (connected for example by flat cable) to the first accelerometer sensor (all possibly improved by a container made of soft material such as rubber or silicone to further decouple the signals) would allow an improved detection of the breathing process.

In fact, the second accelerometer sensor would not perceive the vibrations due to the baby's breathing and/or heartbeat, but would perceive the external vibrations, for example, connected to the wheelchair or car's motion. An appropriate combination of the two signals received (for example a difference) would allow recognizing the breathing (and/or the heartbeat) even in case of unwanted vibrations.

In this regard, the support 2 may provide for the presence of three distinct accelerometer sensors of which one is configured for allowing the determination of the heartbeat and two configured for allowing the determination of the breathing presence/rate.

The wearable support 2 comprises at least one position sensor 14, in particular a geographical position sensor. The position sensor defines a global positioning system, in which the position sensor 14 is a GPS (Global Positioning System) sensor configured for emitting a signal representative of the geographical position of the support 2. The control unit 19, in particular the first controller 19a, is configured for receiving at least at least one signal, emitted by the position sensor 14, representative of the position of the support 2 of the detecting device 1. Also, the control unit 19, in particular the first controller 19a, is configured for calculating a value representative of the position of the support 2 of the detecting device, in particular a value representative of the position in Cartesian or polar coordinates of the support 2 of the detecting device. In greater detail, the value representative of the position has a tolerance of less than 30 m, in further detail a tolerance of less than 10 m, in particular with a tolerance of less than 1 m.

The control unit 19, in particular the first controller 19a, is configured for receiving as an input the signals emitted by the first and second temperature sensors 10, 11, by at least one biometric sensor 12, by at least one accelerometer sensor 13 and at least one position sensor 14: in detail, the control unit 19, in particular the first controller 19a, is configured for receiving said signals at the same time or out of phase over time, as well as for carrying out the processing adapted to define the respective values, in parallel or in series.

The wearable support 2 comprises at least one first temperature sensor 10 configured for emitting at least one signal representative of a temperature at the first surface 2a of the support 2. Optionally, the first temperature sensor 10 may be placed at the first surface 2a of the support 2, in particular the first body temperature sensor 10 may be facing the first surface 2a of the support 2. In an optional embodiment, the support 2 may comprise more than one temperature sensor 10.

The support 2 further comprises at least a second room temperature sensor 11 configured for emitting at least one signal representative of a temperature at the second surface 2b of the support 2. Optionally, the second room temperature sensor 11 may be placed at the second surface 2b of the support 2, in particular the second temperature sensor 11 may be facing the second surface 2b of the support 2. In an optional embodiment, the support 2 comprises more than one temperature sensor 11.

The first and second temperature sensors 10, 11 may be of the resistive type, in which the temperature variation determines a measurable variation of an electrical parameter, in particular of the electrical resistance of a conductor or semiconductor of the temperature sensor itself, or a variation of an electric voltage.

Optionally, the first and second temperature sensors 10, 11 may be of the optical, laser, infrared type or based on a piezoelectric effect.

The control unit 19 is configured for receiving as an input the signals emitted by the first and second temperature sensors 10, 11. In detail, the first controller 19a is configured for receiving as an input the signals emitted by the first and second temperature sensors 10, 11. Moreover, the control unit 19, optionally the first controller 19a of the support 2, is configured for estimating a value representative of a body temperature of the user 3 as a function of both the signals emitted by the first and second temperature sensors 10, 11. In other words, the temperature sensor 10 in contact with the user detects a surface temperature of the user, which is different from (and typically below) the (internal) body temperature of the user, suffering from the effects of the room temperature to which the portion of the body of which the temperature is detected is subjected. Therefore, the internal temperature value of the user is determined by a function of the external body temperature detected by the sensor 10 suitably corrected on the basis of the room temperature detected by the sensor 11.

Moreover, the control unit 19, optionally the first controller 19a of the support 2, is configured for estimating a value representative of a room temperature as a function of both the signals emitted by said the first and second temperature sensors 10, 11. Also in this case, in fact, temperatures are influenced, meaning that the room temperature sensor 11, although not in contact with the skin of the user, is influenced by the heat emitted by the latter being in a position close to the user him/herself (while being well insulated, the room temperature sensor 11 detects an air temperature in the vicinity of the user's body and the latter will inevitably be influenced by the heat of the user him/herself. Therefore, a correction of the measured value that allows obtaining a more reliable room temperature value is taken into account.

The control unit 19 (for example the first controller 19a) is configured for determining the temperature values at the first and second surface 2a, 2b as a function of the signals emitted by the first and second temperature sensors 10, 11.

The value representative of a body temperature of the user 3 is therefore estimated by multiplying the temperature value at the first surface 2a by a coefficient dependent on the temperature value at the second surface 2b of the support 2.

In other words, the value representative of the body temperature of the user 3 is influenced by the external temperature, in particular by the room temperature: in order to provide an accurate measurement of the body temperature, the value representative of the temperature at the surface 2a facing the surface body of the user 3 is compensated as a function of the temperature measured at the second surface 2b of the support 2. In other words, the measurement provided by the first temperature sensor 10 is compensated taking into account the measurement provided by the second temperature sensor 11. in particular, the value representative of the body temperature of the user 3 is estimated by multiplying the temperature value at the first surface 2a by a coefficient dependent on the temperature value at the second surface 2b.

In this regard, the described solution may be implemented by using a real function that receives the signal detected by the first and second temperature sensors and provides the body temperature value as an output. The function may be a continuous or discontinuous function and may be determined experimentally by data interpolation.

Alternatively, the control unit 19 may be provided with an experimentally obtained two-dimensional table with discrete values, such that, given the two inputs, the resulting value is the patient's internal body temperature. The two inputs may be the signals of the first and second temperature sensors, i.e. the surface body temperature and the room temperature detected.

It should however be noted that the mentioned correction coefficient may also be a function of the time history of the signals emitted by the first and second temperature sensors 10, 11: in particular, said coefficient may take into account the time variation rate of the signal emitted by the first and/or by the second temperature sensor 10, 11. This further implementation allows taking into account specific situations such as an anomalous and sudden heating of the room temperature (due for example to having forgotten the child inside a vehicle), however providing a more correct value of the patient's internal temperature, but activating a different alarm routine.

Furthermore, said coefficient may be a function of the signals and/or values obtained by the biometric sensors 12, by the accelerometer sensor 13, by the GPS position sensor 14 or a combination thereof. For example, an accelerated heartbeat accompanied by accelerometer sensor signals that indicate a situation in which the user performs physical activity may result in a different corrective coefficient that takes into account a certainly higher body surface temperature. Vice versa, the presence of an accelerated heartbeat without physical activity may actually mean a feverish situation and confirm (and optionally correct) the reading of the temperature sensors in this sense.

Finally, the device can be appropriately ‘calibrated’; in fact, once the device is worn and all the sensor readings are stabilized, it is possible to communicate a surface temperature and external temperature reading detected with a different instrument in a reliable manner. This ‘calibration’ allows associating the read value of the signal of the sensors mounted on the support 2 with an effective and reliable temperature value so as to correct the initial reading in an instantaneous and reliable manner.

In an embodiment that can be alternative (or combined) to the previous one, the control unit 19 may estimate the value representative of the internal body temperature of the user as a function of the variation of the signal emitted by the first temperature sensor 10 in a predetermined time interval. In greater detail, the control unit 19 is arranged for receiving from an auxiliary device, such as an external thermometer, at a time instant T, an internal body temperature value measured at the same instant T on the user; at this point, the value of the internal body temperature of the user 3 is estimated by the control unit 19 on the basis of the signal from the first temperature sensor 10 corrected according to the internal body temperature value received from the external thermometer.

In this way, the device will suffer less from errors related to an internal body temperature measurement made exclusively by means of the first temperature sensor 10, a measurement made complex due to various parameters that may influence it such as lack of or poor sensor contact, sweat, presence of clothes, etc. The initial detection made through the first temperature sensor is in fact associated (corrected or replaced) to the correct internal temperature measurement (due to having received this reliable measurement by means of an external thermometer with reliable detection). At this point, the temporally subsequent temperature readings will be used to determine a ‘delta’ (for example an increase of 1° C.) with respect to the initial situation received from the device, known and reliable (for example 36.6° C.) or, the difference in temperature between the initial condition and the temporally subsequent condition detected by means of the first temperature sensor will be linked to the absolute temperature received from the external device allowing a more reliable detection of any feverish situation (in the specific example determining a temperature of 36.6°+1°=37.6° C.).

The same procedure may be used to estimate the value representative of the room temperature as a function of the variation of the signal emitted by the second temperature sensor 11 in the predetermined time interval. Also in this case, the control unit 19 receives, at a time instant T, from an auxiliary device, for example a thermometer, a detected room temperature value at the instant T in the room where the user is located. The value of the room temperature is then estimated by the control unit 19 on the basis of the signal from the second temperature sensor 11 corrected as a function of the room temperature value received. Therefore both the information relating to the actual temperature of the user 3 and that relating to the actual room temperature may be provided by one or more auxiliary devices/thermometers, preferably by remote connection of the Bluetooth, RFID or WiFi type.

In a further significant variant, the control unit 19 could monitor over time the surface body temperature signal determined by the first temperature sensor. Assuming also an erroneous/imperfect initial surface body temperature reading or erroneous/imperfect calculation of the internal body temperature due to the influence of the room temperature on the user's skin, the control unit 19 could only (or in combination with what described above) check temperature variations over time. When the variation in the surface body temperature detected exceeds a certain threshold (e.g. 1.5° C.), the control unit 19 would send an alarm signal. In fact, although the initial detection may not be precise in absolute terms, it is assumed that this initial detection indicates a temperature at which the user is not in a feverish state (this could possibly be confirmed to the control unit). Upward variations (temperature increments) above a certain threshold (possibly confirmed with the continuation of the condition for a time considered sufficient to make the reading reliable) are considered potential alarm situations and are signaled. The user may in fact have a fever or even have been forgotten in an environment that heats up suddenly, for example in a car. In order to make the system more reliable, the control unit 19 could carry out additional checks, such as for example checking the room temperature variations in the same time interval and/or the rate of variation of the temperature (or its derivative).

Exceeding the variation threshold (e.g. +1.5° C.) by the surface body temperature accompanied by the same or greater variation of the room temperature over a short time interval (i.e. a derivative of high temperature) would indicate with higher probability to have forgotten the user in the car. The passing of the threshold by the body temperature variation, in the presence of substantially constant room temperature and with a non-high variation rate would more likely indicate a feverish state.

The wearable support 2 further comprises signal transmitting means 15 (see accompanying figures), wherein said transmitting means are configured for remotely transmitting data. In particular, the transmitting means 15 are configured for remotely transmitting the signals emitted by the first and second temperature sensors 10, 11, the signals emitted by the biometric sensors 12, the signals emitted by the accelerometer sensors 13 and/or the signals emitted by the position sensor 14.

In greater detail and according to a preferred embodiment, the transmission means 15 are configured for remotely transmitting at least one in the group between:
the temperature values at the first and second surfaces 2a, 2b of the support 2;
the value representative of the internal body temperature;
the value representative of a calculated room temperature;
the value representative of the position of the support 2;
the value representative of a movement;
the value representative of the heart rate;
the value representative of the breathing rate;
the value representative of the presence of a breathing process;
the value representative of the blood oxygen concentration.

The signal transmission means provide the use, by way of example and not limited thereto, of a radio frequency transmission system, alternatively of an RFID (Radio-Frequency IDentification) system, optionally of a Wi-Fi system based on the IEEE 802.11 standard specifications, or a Bluetooth system.

In a further embodiment that is not preferred as it increases the costs of the support, the wearable support 2 comprises at least one screen 20 configured for allowing the display of the physiological parameters such as:
the temperature values at the first and second surfaces 2a, 2b;
the value representative of the body temperature;
the value representative of a room temperature;
the value representative of the position of the support 2;
the value representative of a movement;
the value representative of the heart rate;
the value representative of the breathing rate;
the value representative of the presence of a breathing process;
the value representative of the blood oxygen concentration.

Optionally, the screen may be flat or counter-shaped to the surface 2b of the support 2: moreover, it is possible to use a rigid or flexible screen.

The control unit 19, in particular the first controller 19a integrated in the support, is configured for defining one or more confidence intervals with respect to which the values of the parameters of interest detected by means of the plurality of the sensors described above are compared. In an embodiment, the control unit 19 defines one or more predetermined confidence intervals, relative to each representative value of the respective acquired parameter. In other words, it is admitted that each value representing a parameter of interest may vary within the respective confidence interval. As an example, it is admitted that the heartbeat rate may vary within a confidence interval of between 40 and 190 beats per minute. Said predetermined confidence intervals may vary according to age, gender, a possible pathological condition of the user 3, or as a function of another plurality of variables.

In a further embodiment, the control unit 19 defines one or more confidence intervals as a function of the combination of the values representative of the parameters of interest. Optionally, the control unit 19 defines one or more confidence intervals as a function of the interaction between the values representative of the acquired parameters. In other words, the values of a confidence interval in reference to a single parameter of interest, depending on the values representative of the plurality of the parameters of interest. By way of example, the confidence interval referred to the frequency of heartbeats may depend on the values representative of the additional parameters of interests, such as body temperature, external temperature or movement threshold. In said embodiment, the confidence intervals may therefore vary over time as the plurality of the values representative of the parameters of interest vary.

The control unit 19, in particular the first controller 19a, is configured for comparing at least one value representative of one or more parameters of interest with a respective confidence interval and, as a function of this comparison, define:
a safety condition, wherein the representative value falls into the respective confidence interval;
a danger condition, wherein the at least one representative value exceeds the confidence interval.

The danger condition therefore represents a situation in which the values representative of the parameters of interest such as temperature, physiological parameters, presence or absence of movement, value representative of the movement or geographical position are outside a range of values considered of safety. In this danger condition, the control unit 19 or the first controller 19a of the support 2 is configured for emitting a respective alarm signal.

The confidence interval relative to the value representative of the geographical position of the support 2 defines a safety area, in which the control unit 19 or the first controller 19a of the support 2 are configured for comparing the value representative of the geographical position of the support 2 with the confidence interval defining the safety area. Moreover, the control unit 19 or the first controller 19a of the support 2 are configured for defining the danger condition if the confidence interval defining the safety area does not include the value representative of the geographical position of the support 2. The safety area therefore represents an area defined within a perimetral edge within which it is believed that the user of the support 2 is safe. In this regard, the device may be used to determine that the user (for example, the baby) should remain within an area of 50 meters from a receiving device, or should not stray too far from a parent. In this regard, the GPS allows monitoring the position of both the support (and therefore the baby) and the remote device (cell phone of the parent), emitting an alarm on the remote device whenever the distance exceeds the predetermined safety distance. Note that this same function may also be integrated in the absence of a GPS system in the support 2; in fact, it will be possible to determine that the support sends a control signal every predetermined time interval and that the transmission/reception range is a certain maximum distance beyond which the signals are no longer received (e.g. Bluetooth at 10 m). Where the signal is not received for one or more (pre-settable) time intervals, the alarm is emitted. This function, in any of the proposed implementations, allows actively monitoring the proximity or not of the user. Moreover, the position function by means of a sensor in the wearable support also allows cooperating with other functions of the device. In fact, some locations may be defined as safe (for example nursery school). When the control unit 19 detects (for example via GPS) that the user is in a safe place, he/she can deactivate the distance monitoring (and/or other parameters) automatically.

In an optional embodiment, the wearable support 2 comprises at least one vibrating element, such as a vibrodine 16, configured for generating a predetermined vibration. Reference will be made herein to a vibrodine meaning in fact any element capable of generating an appropriate vibration (well perceptible by the user) in the support if electrically stimulated/controlled. In particular, the vibrodine 16 is configured for generating a predetermined vibration in the danger condition defined by the control unit 19, in particular by the first controller 19a. The vibrodine 16 allows generating a predetermined vibration signal, having a set intensity and frequency constant or variable over time according to a predetermined law. Optionally, the vibrodine 16 may generate a different vibration as a function of the parameter(s) of interest which are outside the respective confidence interval. In a further embodiment, the vibrodine 16 is configured for generating a vibration in the safety condition. The control unit 19, in particular from the first controller 19a, is configured for activating and deactivating the vibrodine 16. The vibrodine may also be deactivated via the parent's cell phone. This function is relevant where it may be necessary to attempt to wake up the user or make him/her react. Think, for example, of a prolonged breathing interruption (apnea) at night. The device could (automatically or not), as a further reaction to an alarm proposed by the remote device 100, attempt to awaken the user by generating a vibrational disturbance, for example, to the arm or ankle to make him/her resume the interrupted vital function.

The wearable support 2 may further comprise at least one electroacoustic transducer such as a loudspeaker 17. Hereinafter, reference will be made to any type of electroacoustic transducer by the name of the loudspeaker. Said loudspeaker 17 is configured for emitting a respective sound signal. In particular, the loudspeaker 17 is configured for emitting a sound signal in the danger condition defined by the control unit 19, optionally by the first controller 19a. The loudspeaker 17 allows a predetermined sound signal to be emitted, having a set intensity and frequency constant or variable over time according to a predetermined law. Optionally, the loudspeaker may generate a different sound signal depending on the parameter(s) of interest which are outside the respective confidence interval. In a further embodiment, the loudspeaker is configured for generating a sound signal in the safety condition. The control unit 19, in particular from the first controller 19a, is configured for activating and deactivating the loudspeaker 17. This signal could in a particular embodiment also comprise a voice message coming from the remote device 100.

The wearable support 2 comprises at least one battery 18, preferably rechargeable, supplying the control unit 19 or the first controller 19a and the one or more sensors of the support 2. In a preferred embodiment, the battery 18 supplies at least one of the loudspeaker, the vibrodine, the screen, the GPS position sensor, the signal transmission means, or any other sensor engaged to the support 2.

The detecting device 1 comprises at least one remote external device 100 distinct from the support 2 and optionally distant from the support 2 itself. In a preferred embodiment, the external device 100 may be, by way of example, an electronic device such as a tablet, smartphone, remote computer, a server, a laptop computer. In detail, the external device 100 comprises signal receiving means 101 configured for remotely receiving from the transmission means 15 of the support 2 the values representative of the parameters of interest or the signals emitted by the temperature sensors 10, 11, by the biometric sensors 12, by the accelerometer sensor 13 or by the position sensor 14 of the support 2. In detail, the signal receiving means 101 of the external device are configured for receiving (all) the signals available among:
the value representative of the body temperature of the user;
the temperature values at the first and second surfaces 2a, 2b;
the value representative of a room temperature;
the value representative of the position of the support 2;
the value representative of a movement;
the value representative of the heart rate;
the value representative of the breathing rate;
the value representative of the presence of a breathing process;
the value representative of the blood oxygen concentration;
a respective alarm signal emitted by the control unit 19 of the detecting device 1, in particular by the first controller 19a of the support 2, for detection during the danger condition.

The control unit 19 comprises a second controller 19b engaged to the external device 100 and placed in connection with at least the signal receiving means 101. For descriptive purposes of the technical features of the controller 19b, reference may be made to what has been described above with reference to the control unit 19 of the detecting device 1 and to the first controller 19a of the support 1.

In this regard, all the functions that have been described in relation to the control unit 19 may be performed by the first and/or the second controller 19a, 19b depending on the configuration of the device. In other words, the processing functions of the signals received by the sensors on the support 2 may be carried out where deemed most appropriate, that is directly by the first controller 19a integrated in the support 2 and/or by the second controller integrated in the remote device 100, without departing from the innovative concepts of the present embodiments.

The second controller 19b engaged to the external device 100 is configured for receiving as an input, from the signal receiving means 101, the values representative of the parameters of interest or the signals emitted by the temperature sensors 10, 11, by the biometric sensors 12, by the accelerometer sensors 13 or by the position sensor 14 of the support 2.

In an embodiment in which the first controller 19a engaged to the support 2 is not configured for estimating the value representative of a body temperature of the user 3, the second controller 19b is configured for receiving as an input at least one in the group of:
the temperature values at the first and second surfaces (2a, 2b);
the signals emitted from the first and second temperature sensors (10, 11).

In said embodiment, the second controller 19b is thus configured for determining a value representative of a body temperature of the user 3 as a function of both the signals emitted by the first and second temperature sensors 10, 11. Moreover, the second controller 19b is configured for estimating a value representative of a room temperature according to both the signals emitted by said first and second temperature sensors 10, 11. In addition, the second controller 19b is configured for determining the temperature values at the first and second surfaces (2a, 2b).

In other words, the analysis of the signals provided by the plurality of sensors of the support 2 may be carried out:
by the first controller 19a, according to a first embodiment, engaged to the support 2, thus allowing an “on-site” analysis and management of the signals themselves without the need for a connection between the transmission means 15 of the support 2 and the signal receiving means 101 of the external device 100;
by the second controller 19b, according to a second embodiment, engaged to the external device 100, so as to lighten the work of the first controller 19a engaged to the support 2 and allow a longer life of the battery of the support 2 or a reduction of the weight of the support 2 if one decides to reduce the size of the battery.

According to the first embodiment, the second controller 19b is further configured for receiving as an input the temperature values at the first and second surfaces 2a, 2b of the support 2 and the value representative of the body temperature of the user. According to the same embodiment, the second controller 19b is configured for receiving as an input, from the signal receiving means 101 of the external device 100, at least one value representative of the physiological parameters detected by one or more of the biometric sensors 12 of the support 2. Furthermore, the second controller 19b is configured for receiving as an input, from the signal receiving means 101 of the external device 100 a value representative of an acceleration of the support 2. Again according to said first embodiment, the second controller 19b is configured for receiving as an input, from the signal receiving means 101 of the external device 100 a value representative of a position of the support 2.

According to the second embodiment, the second controller 19b is configured for receiving at least at least one signal representative of one or more physiological parameters of the user 3 emitted by the biometric sensors 12 and for determining at least one value representative of the respective physiological parameters. According to the same embodiment, the second controller 19b is configured for receiving as an input a signal representative of an acceleration emitted by the accelerometer sensor 13 of the support 2 and for determining a value representative of an acceleration of the support 2. Moreover, the second controller 19b is configured for receiving as an input a signal representative of a position of the support 2 emitted by the position sensor 14 of the support 2, and for determining a value representative of a geographical position of the support 2. According to the second embodiment, the second controller 19b is configured for defining one or more confidence intervals, defined in the same way described above. The second controller 19b is thus configured for:

comparing at least one value representative of one or more parameters of interest with a respective confidence interval;

and, as a function of said comparison:
defining a safety condition, wherein the representative value falls into the respective confidence interval;
defining a danger condition, wherein the representative value exceeds the confidence interval.

The second controller 19b is thus configured for emitting an alarm signal in the danger condition.

The external device comprises a further position sensor 102, optionally a position sensor 102 defining a global positioning system. In particular, the position sensor 102 is a GPS (Global Positioning System) sensor configured for emitting a signal representative of the geographical position of the external device 100. Optionally, the position sensor 102 defines a GNSS (Global Navigation Satellite System) positioning system. The second controller 19b is thus configured for receiving as an input the signal representative of the position of the external device 100 emitted by the geographic position sensor 102 and for determining and/or calculating a value representative of the geographical position of the external device. In detail, the second controller 19b is configured for determining a value representative of the position in Cartesian or polar coordinates of the external device 100, so as to have a tolerance of less than 30 m, optionally a tolerance of less than 10 m, in particular with a tolerance of less than 1 m.

The confidence interval relative to the value representative of the geographical position of the external device 100 defines a relevance area, in which the control unit 19 or the second controller 19a of the external device are configured for comparing the value representative of the geographical position of the support 2 with the confidence interval defining the relevance area. In addition, in which the control unit 19 or the second controller 19a of the external device are configured for defining the danger condition if the confidence interval defining the relevance area does not include the value representative of the geographical position of the support 2. In other words, the relevance area defines an area bounded by a perimetral edge and comprising the value representative of the geographical position of the external device 100. If the support 2, in particular the value representative of the support 2, are not included within the relevance area, the control unit 19 or the second controller 19b are configured for defining the danger condition, as the support 2 would be at a greater distance from the one established by said relevance area. In other words, the relevance area may define an area limited to a maximum distance from the external device 100.

In one embodiment in which the support 2 does not comprise any position sensor, the position of said support 2 may in any case be estimated by means of the external device 100: in particular, the external device is configured for evaluating the presence of the support 2 within a predetermined distance (for example, because it receives signals from the support and these signals can be received at most at a distance of a few meters). By way of example, therefore, if the external device 100 detects the presence of the support (receiving signals from the latter) and also that the position in which the device 100 itself is located is known/safe (in which, for example, the user in at the nursery school), it can determine that the position of the support 2 is also safe; the device 100 itself disables any alarm messages concerning the position of the support 2 when the wireless contact is subsequently lost as it is determined that the user is in a place considered safe.

The external device 100 comprises at least one screen 105 configured for displaying one or more values representative of the parameters of interest. The screen 105 is therefore adapted to allowing a reference user to display the values of the individual parameters of interest, possible interactions between them, the presence or absence of a dangerous condition, the trend over time of the values representative of the parameters of interest. Moreover, the screen 105 is configured for displaying the geographical position of the support 2 and/or of the external device 100, so as to highlight the mutual distance.

The external device 100 may comprise at least one further vibrodine 103 configured for generating a predetermined vibration. In particular, the second controller 19b is configured for activating the further vibrodine 103 to emit a vibration. In greater detail, the second controller 19b is configured for activating the further vibrodine 103 for emitting a vibration in the danger condition defined by the first controller 19a and/or the second controller 19b.

The further vibrodine 103 may generate a different vibration as a function of the parameter(s) of interest which are outside the respective confidence interval. In a further embodiment, the further vibrodine 103 is configured for generating a vibration in the safety condition. The control unit 19, in particular the second controller 19b, is configured for activating and deactivating the further vibrodine 103.

The external device 100 comprises at least one further loudspeaker 104 configured for emitting a sound signal. In particular, the further loudspeaker is configured for emitting a sound signal in the danger condition defined by the first controller 19a and/or by the second controller 19b.

Optionally, the further loudspeaker 104 may generate a different sound signal according to the parameter(s) of interest which are outside the respective confidence interval. In a further embodiment, the loudspeaker 104 is configured for generating a sound signal in the safety condition. The control unit 19, in particular the second controller 19b, is configured for activating and deactivating the further loudspeaker 104.

The external device 100 further comprises storage means configured for recording the values representative of the parameters of interest, in particular the values of the parameters of interest received by means of the signal receiving means 101 or the values of the parameters determined by the second controller 19b of the control unit 19.

Given the foregoing described mainly from the structural point of view, the device thus conceived may carry out a plurality of tasks and monitoring to perform functions of a different nature.

Upon application to the wrist or ankle of (for example) the baby, the device begins to process and filter the data of the biometric sensors to process (presumably in about 1 minute):
the surface temperature (skin) of the baby and develop a corrective action that takes into account the room temperature to determine the body temperature;
the breathing rate (for example, by suitably filtering it and recognizing the vibrations with respect to those of the heartbeat, with respect to those of the movement of the baby, with respect to those of external disturbances);
the heartbeat.

In the event that the breath, the heat beat and/or the pressure increase abnormally (and due to previously studied situations due to the baby crying in his/her bedroom), the remote device 100 (for example the smartphone) warns the parents (who may be in another room), with an appropriate acoustic and/or visual signal, perhaps with a pre-recorded crying-like alarm.

Under normal conditions, i.e. in the absence of abnormal situations or of presumed danger, the wearable support 2 sends regular information to the remote device 100 indicating that there are no problems to report.

In the absence of breath and/or lack of heat beat and/or reduced pressure (particularly in the event of a combination of these situations), the wearable support 2 sends an immediate alarm signal to the remote device 100 (via Bluetooth or wireless). At the same time, it activates the internal vibrodine of the support 2 to try to wake up the baby, if necessary also at the maximum power taking into account the severity of the detected conditions.

It may then again be provided for more safety that if the remote device 100 does not receive one or more of these signals it give an alarm, since the cause could be that the baby has stopped breathing, the heartbeat has stopped, the pressure has dropped dramatically, but it may also be an anomaly in the contact or in the wearable support 2, a low battery of the support, the baby forgotten in the car, or the baby who has strayed too far from the device 100 (e.g. smartphone) of the parents.

Of course, if the baby's temperature rises, the alarm is sent remotely. It could be due to fever, or to the baby forgotten in the car in an environment that is rapidly heating up (the wireless connection is still active). In this case also the room temperature, i.e. the car, rises, giving further confirmation of the dangerous situation.

When the remote device 100 recognizes (via GPS or GNSS) at predetermined times a place determined as “safe” (the nursery, home, etc.), it may send a message to a further smartphone indicating that the baby “has been delivered” and can automatically disable any alarms.

If there is not the expected passage to a safe place, the system obviously remains active and when the remote device 100 is no longer receiving the signal, it gives an immediate alarm.

For optimal control, the support/bracelet should always be worn and removed only occasionally to allow recharging thereof. It will be possible to provide that it is waterproof or suitably insulated in order not to make it necessary to remove it in particular situations, for example during a bath, in the pool or on the beach. If necessary, the battery of the support 2 may be charged wirelessly.

In addition to the above, if the wearable support/bracelet is removed, the remote device 100 no longer receives data on the vital parameters and then gives the alarm. In this case, the alarm may be temporarily deactivated by the remote device 100 and if within a certain time the bracelet is not worn again, the alarm resumes. This time could be variable and stretch appropriately and the alarm could become even a reminder message in case the bracelet battery was being recharged.

In summary, the device may have different functions (selectively activable) and via the remote device 100 such as a smartphone to warn in the following cases:
Fever: if the temperature (and possibly the heartbeat) of the baby increase beyond predetermined limits;
SIDS: if the breath and/or heartbeat cease and/or if the pressure drops drastically;
ALTE (Apparent Life-Threatening Events): such as prolonged apneas during sleep;
DoNotForgetMe: if the baby is forgotten in the car (for example, an anomalous increase in the room temperature or the baby's or the absence of a signal if the car is far away);
Crying: if the baby cries in his/her bedroom (by combining parameters values such as breathing, movement, heartbeat, etc.);
Distance: if the child has strayed from the parents;
Monitoring: recording on the remote device or smartphone of the trend of sleep on the previous nights (including highlighting any apneas, crying, or other anomalies, etc.

Even if disconnected from any remote device or smartphone, the wearable support 2 can continue to detect the child's parameters (compatibly with the duration of the battery charge), so that they can be synchronized at the first connection so as to also monitor the progress of the health of the user when he/she is not under direct control or monitoring, even just to check for example if he/she has slept, cried, etc.

Of course, the monitoring device, although particularly suitable for babies, could also be used for the elderly or disabled or in any case for any person who needs to be subjected to one or more of the control or monitoring functions mentioned above.

The wearable support, if appropriately queried in conditions of course of safety for the user and without disclosing sensitive information if not to authorized parties, could also provide a contact telephone number and/or data of the user, such as personal data, address, blood group and any other useful information in case the baby (or elderly or disabled) gets lost and/or has an accident.

The version of the support equipped with GPS and/or GNSS (Global Navigation Satellite System) will eventually allow identifying users that have been lost, transmitting the position data for example on query or even tracing the bracelet that has detached or that has been fraudulently taken.

In order to protect sensitive data and also act as a deterrent to theft, the wearable support will only work in conjunction with remote devices or smartphones registered upon the first activation and subsequent remote devices added thereafter only with the permission of the former. Likewise, one or more external devices 100, such as smartphones or tablets or computers, may interact with multiple wearable detecting devices 1, for example in the case of nursery in the hospital or nursery schools.

1-17. (canceled)

18. A device to detect parameters of a user, comprising:
a support wearable by the user and exhibiting a first surface configured for contacting a body surface of the user,
said support exhibiting at least one second surface distanced from, and opposite to, the first surface,
the wearable support comprising:
at least one first temperature sensor configured for emitting at least one signal of a temperature at the first surface of the support, and
at least one second temperature sensor configured for emitting at least one signal of a temperature at the second surface of the support,
a control unit configured to:
receive as an input at least the signals emitted by the first and second temperature sensors,
estimate a value representative of an internal body temperature of the user as a function of both the signals emitted by said first and second temperature sensors.

19. The device according to claim 18, wherein the control unit is configured to estimate a value representative of a room temperature as a function of both the signals emitted by said first and second temperature sensors.

20. The device according to claim 18, wherein the value representative of an internal body body temperature of the user is estimated by multiplying the temperature value at the first surface by a coefficient dependent on the temperature value at the second surface of the support, or wherein the value representative of an internal body temperature of the user is estimated by using a two-input prestored table, the inputs being the surface body temperature determined as a function of at least the signal emitted by the first sensor temperature and the room temperature determined as a function of at least the signal emitted by the second temperature sensor, the output value being the internal body temperature of the user.

21. The device according to claim 18, wherein the control unit is configured for:
receiving as an input at least the signal emitted by the first temperature sensor,
determining a first magnitude as a function of the variation of the signal emitted by the first temperature sensor in a predetermined time interval,
comparing said first magnitude with at least one first threshold,
outputting, as a function of said comparison, a respective alarm signal.

22. The device according to claim 21, wherein the control unit is configured for:
receiving as an input at least the signal emitted by the second temperature sensor,
determining a second magnitude as a function of the variation of the signal emitted by the second temperature sensor in a predetermined time interval,
defining the first threshold as a function of said second magnitude.

23. The device according to claim 21, wherein the control unit is configured for:
receiving as an input at least the signal emitted by the second temperature sensor,
determining a second magnitude as a function of the variation of the signal emitted by the second temperature sensor in a predetermined time interval,
comparing said second magnitude with at least one second threshold,
outputting, as a function of said comparison of the first magnitude with the first threshold and of the second magnitude with the second threshold, a respective alarm signal.

24. The device according to claim 18, wherein the control unit is configured for:
receiving, at a time instant T, from a thermometer, an internal body temperature value detected at the instant T′ on the user,
determining a value of the internal body temperature of the user based on the signal from the first temperature sensor, said temperature value being corrected as a function of the internal body temperature value received by the auxiliary device;
receiving, at a time instant T, from a thermometer, a detected room temperature value at the instant T′ in the environment wherein the user is located,
determining a value of the room temperature based on the signal coming from the second temperature sensor, said temperature value being corrected as a function of the room temperature value received from the auxiliary device.

25. The device according to claim 18, wherein the wearable support further comprises one or more sensor among:
a sensor configured for emitting a signal representative of the breathing rate;
a sensor configured for emitting a signal representative of the presence or absence of a breathing process;
an accelerometer sensor configured for emitting at least one signal representative of a movement;
wherein the control unit is configured for:
receiving as an input at least the signals emitted by at least one sensor,
determining, as a function of the input signals, a signal representative of the breathing rate or a signal representative of the presence or absence of a breathing process.

26. The device according to claim 18, wherein the wearable support comprises at least one vibrodine configured for generating a predetermined vibration, the control unit selectively actuating the vibrodine after determining the signal representative of the breathing rate or the signal representative of the presence or absence of a breathing process, the control unit actuating the vibrodine in absence of a breathing process or in the presence of a breathing rate less than a preset safety value.

27. The device according to claim 18, wherein the wearable support comprises one or more biometric sensors, said biometric sensors being configured for emitting one or more signals representative of one or more physiological parameters of the user detectable at the first surface of the support,
wherein the control unit is configured for:
receiving as an input said one or more signals representative of physiological parameters;
determining one or more values representative of the respective physiological parameters; and wherein the one or more biometric sensors comprise at least one in the group among:
at least one sensor configured for emitting a signal representative of the heart rate;
at least one sensor configured for emitting a signal representative of the breathing rate;
at least one sensor configured for emitting a signal representative of the presence or absence of a breathing process;
at least one sensor configured for emitting a signal representative of the blood oxygen concentration;
at least one sensor configured for emitting a signal representative of the blood CO₂ concentration;
the control unit being configured for receiving as an input at least one among:
a signal representative of the heart rate;
a signal representative of the breathing rate;
a signal representative of the presence of a breathing process;
a signal representative of the blood oxygen concentration;
a signal representative of the blood CO₂ concentration;
said control unit being configured for calculating respectively at least one among:
a value representative of a time heart rate;
a value representative of the time breathing rate;
a value representative of the presence of a breathing process;
a value representative of the blood oxygen concentration;
a value representative of the blood CO₂ concentration.

28. The device according to claim 18, wherein the wearable support comprises at least one accelerometer sensor configured for emitting a signal representative of at least one between the rate and the presence or absence of a breathing process of the user.

29. The device according to claim 28, wherein the wearable support further comprises an auxiliary accelerometer sensor, the control unit being configured for receiving the signals from the accelerometer sensors and for processing signals together for obtaining at least one signal representative of at least one between the rate and the presence or absence of a heartbeat of the user, the accelerometer sensor being positioned at the first surface of the wearable support for contacting the user when the device is in use, the auxiliary accelerometer sensor being positioned on a support distanced from the first surface and not rigidly coupled to the accelerometer sensor, the signal processing being responsible for eliminating or reducing the interferences due to undesired external vibrations.

30. The device according to claim 18, further comprising at least one blood saturation/oxygenation sensor configured for emitting at least one signal representative of the blood saturation/oxygenation in the user or a blood CO₂ concentration sensor configured for emitting at least one signal representative of a blood CO₂ concentration in the user, said sensor being distinct from the support and connectable by a cable or wirelessly to the control unit.

31. The device according to claim 28, wherein the control unit is configured for storing the signal representative of at least one between the rate and presence or absence of a breathing process in the user in a memory connected to the control unit itself, and for graphically representing and saving in a memory the graph of the time trend of the signal representative of at least one between the rate and presence or absence of a breathing process in the user on a display connected to the control unit itself.

32. The device according to claim 18, wherein the wearable support comprises signal transmission means, said transmission means being configured for remotely transmitting at least one in the group among:
the temperature value at the first surface of the support;
the temperature value at the second surface of the support;
a value representative of the body temperature;
a value representative of a room temperature;
a value representative of the geographic position of the support;
a value representative of a movement;
a value representative of the heart rate;
a value representative of the breathing rate;
a value representative of the presence of a breathing process;
a value representative of the blood oxygen concentration;
a value representative of the blood CO₂ concentration.

33. The device according to claim 18, wherein the control unit is configured for defining one or more confidence intervals,
said control unit being further configured for:
comparing at least one value representative of one or more parameters of interest with a respective confidence interval;
as a function of said comparison, defining:
a safety condition, wherein the at least one representative value falls into the respective confidence interval;
a danger condition, wherein the at least one representative value exceeds the confidence interval;
said control unit being configured for emitting, in the danger condition, a respective alarm signal.

34. The device according to claim 32, comprising at least one external device distanced from the support, said external device comprising:
signal receiving means configured for receiving remotely from the transmission means of the wearable support at least one in the group among:
the value representative of the body temperature of the user;
the temperature value at the first surface of the support;
the temperature value at the second surface of the support;
the value representative of a room temperature;
the value representative of the position of the support;
the value representative of a movement;
the value representative of the heart rate;
the value representative of the breathing rate;
the value representative of the presence of a breathing process;
the value representative of the blood oxygen concentration;
the value representative of the blood CO₂ concentration;
a respective alarm signal emitted by the control unit of the detecting device during the danger condition,
the control unit comprising at least one second controller engaged with the external device and connected at least to the signal receiving means of said external device, said second controller being configured for receiving as an input at least one in the group among:
the value representative of the body temperature of the user;
the temperature values at the first and second surfaces of the support;
the signals emitted from the first and second temperature sensors;
said second controller being configured for determining at least one in the group between:
a value representative of a body temperature of the user as a function of both the signals emitted by said first and second temperature sensors, and estimating a value representative of a room temperature as a function of both the signals emitted by said first and second temperature sensors;
the temperature values at the first and second surfaces.

35. A device for detecting parameters of a user, is provided, comprising:
a wearable support wearable by the user and exhibiting a first surface configured for facing and contacting a body surface of the user,
said support exhibiting at least one second surface distanced from, and opposite to, the first surface,
the wearable support comprising a transmitter configured for remotely transmitting data and at least one of:
at least one first temperature sensor configured for emitting at least one signal of a temperature at the first surface of the support, and
at least one second temperature sensor configured for emitting at least one signal of a temperature at the second surface of the support,
a sensor configured for emitting a signal representative of the breathing rate;
a sensor configured for emitting a signal representative of the presence or absence of a breathing process;
an accelerometer sensor configured for emitting at least one signal representative of a movement;
an external receiving device spaced from the support comprising a receiver configured for receiving remotely from the transmitter of the wearable support
at least one control unit configured for:
receiving as an input at least the signals emitted by at least one sensor,
determining, as a function of the input signals, a signal representative of the breathing rate or a signal representative of the presence or absence of a breathing process,
verifying whether the value representative of the breathing rate exceeds or not a predetermined threshold value for the breathing rate,
emitting, in a dangerous condition, a respective alarm signal,
said control unit comprising a first controller engaged to the wearable support and in connection with the transmitter and at least one of the sensors, and a second controller engaged to the external device and in connection at least with the receiver of said external device,
wherein the second controller is configured for emitting the respective alarm signal in the dangerous condition.

36. The device according to claim 35, wherein the second controller being configured for receiving as an input one or more parameters in the group between:
a value representative of the body temperature of the user;
a temperature values at the first and second surfaces of the support;
signals emitted from the first and second temperature sensors;
signals emitted from the sensor configured for emitting a signal representative of the breathing rate;
signals emitted from the sensor configured for emitting a signal representative of the presence or absence of a breathing process;
the signals emitted from the accelerometer sensor;
and second controller being further configured for:
comparing at least one value representative of one or more parameters of interest with a respective confidence interval;
as a function of said comparison, defining:
a safety condition, wherein the at least one representative value falls into the respective confidence interval;
the dangerous condition, wherein the at least one representative value exceeds the confidence interval.

37. The device of claim 36, wherein, the external device comprises:
a screen configured for displaying one or more values representative of the parameters of interest;
a loudspeaker configured for emitting a sound signal;
a vibrodine configured for generating a predetermined vibration.
